(19) United States
(12) Patent Application Publication
(10) Pub. No.: US 2004/0034406 A1
(43) Pub. Date: Feb. 19, 2004

(54) Vascular stent grafts

(76) Inventor: Jeffrey J. Thramann, Longmont, CO (US)

Correspondence Address: HOLLAND & HART, LLP, 555 17TH STREET, SUITE 3200, DENVER, CO, 80201, US

(21) Appl. No.: 10/643,554

(22) Filed: Aug. 18, 2003

Related U.S. Application Data
(60) Provisional application No. 60/404,343, filed on Aug. 19, 2002.
(60) Provisional application No. 60/404,344, filed on Aug. 19, 2002.

(51) Int. Cl. A61F 2/06

(52) U.S. Cl. 623/1.13

(57) ABSTRACT

The present invention provides a vascular stent graft. The vascular stent graft comprises a main vessel stent graft and a bypass stent graft to bypass a portion of vascular anatomy, connecting branch stent graft to provide flow to branch vessels to remove a portion of the vascular anatomy from circulation. The main vessel stent graft is designed with or punctured to contain an access port through which the bypass or connecting branch stent graft is received. A sealing relationship is formed between the stents to prevent leakage or bleeding.

This application claims priority to U.S. Provisional Patent Application Serial No. 60/404,343, filed Aug. 19, 2002, titled MODULAR RECONSTRUCTABLE ENDOVASCULAR BYPASS STENT GRAFT and U.S. Provisional Patent Application Serial No. 60/404,344, filed Aug. 19, 2002, titled MODULAR RECONSTRUCTABLE STENT GRAFT.

### FIELD OF THE INVENTION

The present invention relates to vascular surgery and, more particularly, vascular stent grafts useful in bypassing or removing portions of vascular anatomy from circulation and/or reconstructing vascular anatomy.

### BACKGROUND OF THE INVENTION

The circulatory system comprises many different parts, one of which is the vascular system. Blood vessels can develop various problems, diseases, or other pathology that frequently requires surgical repair.

Two common conditions include vascular blockage, such as, for example, blood clots, and aneurysms. Blockage is generally repaired surgically by, for example, bypass surgery, a balloon catheter, or the like. Surgeons conventionally treat aneurysms by surgically removing the aneurysm. Some aneurysms can be treated using endovascular methodologies including placing a graft, but frequently endovascular treatment is not possible because branch vessels become occluded. But, these and other conventional procedures for correcting vascular pathology are not particularly satisfactory. Thus, it would be desirous to develop apparatuses that allowed for endovascular repair of the vascular system.

### SUMMARY OF THE INVENTION

To attain the advantages and in accordance with the purpose of the invention, as embodied and broadly described herein, apparatuses to facilitate endovascular repair of a diseased vessel are provided. In particular the vascular stent graft comprises a main vessel stent graft and a bypass stent graft. The bypass stent graft has a proximate end to be received in an access port on the wall of the main vessel stent graft. The bypass stent graft has a distal end to be positioning in a sealing relationship with the vessel such that vascular anatomy is bypassed.

The present invention also provides for vascular stent grafts to remove portions of the vascular system from circulation. The vascular stent graft comprises a main vessel stent graft and a branch connecting stent graft. The branch connecting stent graft is received an access port on the wall of the main vessel stent graft such that the distal end of the branch connecting stent graft is in the branch vessel and the proximate end of the branch connecting stent graft is in a sealing relationship with the access port.

The present invention further provides for vascular stent grafts to remove portions of the vascular system from circulation. The vascular stent graft comprises a main vessel stent graft, a branch stent graft, and a connecting stent. The branch stent is placed in the branch vessels and has radiopaque markers for later location. The main vessel stent graft is placed temporarily occluding the branch vessel. A connecting stent is aligned using the radiopaque markers such that the connecting stent has a distal end in sealing relationship with the branch stent graft and a proximate end in sealing relationship with the main vessel stent graft.

The foregoing and other features, utilities and advantages of the invention will be apparent from the following more particular description of a preferred embodiment of the invention as illustrated in the accompanying drawings. Further, the advantages and purpose of the invention will be realized and attained by means of the elements and combinations particularly pointed out in the appended claims.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 shows a portion of a vascular anatomy with an endovascular stent graft consistent with the present invention;

FIG. 2 shows devices useful for placement of the endovascular stent graft of FIG. 1

FIG. 3 shows puncturing a main vessel stent graft consistent with establishing a working port;

FIG. 4 shows a cross-sectional view of an access port and bypass stent graft consistent with an embodiment of the present invention;

FIG. 5 shows a portion of a vascular anatomy with an endovascular stent graft consistent with another embodiment of the present invention;

FIG. 6 shows puncturing a main vessel stent graft consistent with establishing a working port;

FIG. 7 shows a portion of a vascular anatomy with an endovascular stent graft consistent with another embodiment of the present invention; and

FIG. 8 shows another construction of main vessel stent graft 508.

### DETAILED DESCRIPTION

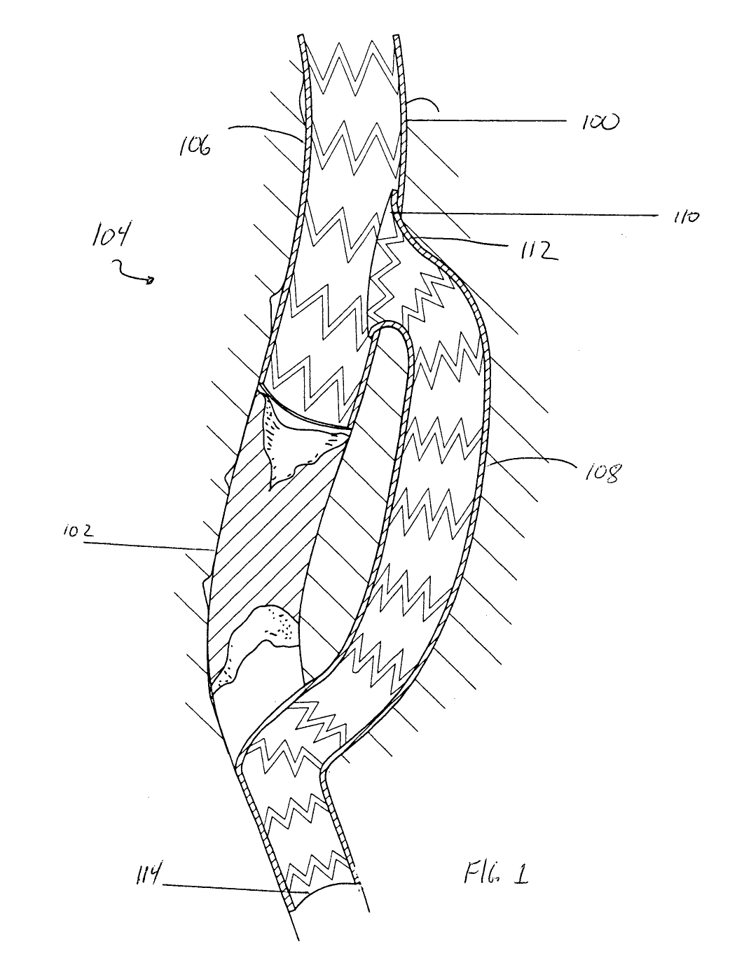

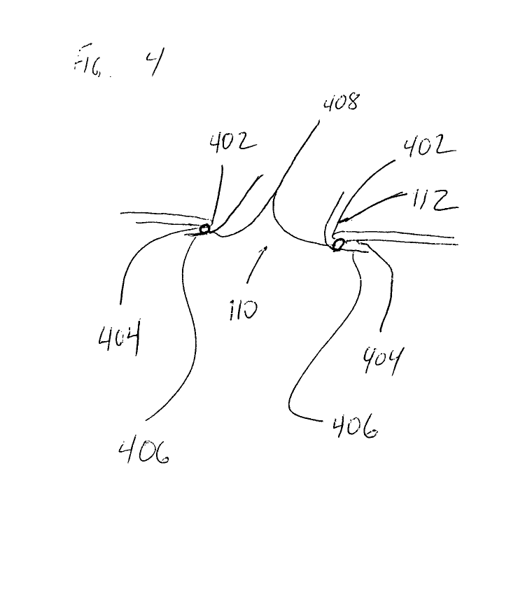

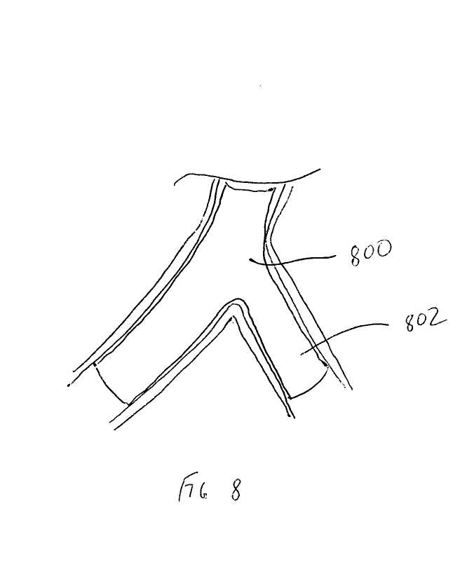

Some embodiments of the present invention are described with reference to FIGS. 1 to 8. Referring first to FIG. 1, a cut-away portion of a blood vessel 100 is shown. A clot 102, blockage, or other vascular pathology in blood vessel 100 requires a bypass. An endovascular bypass stent 104 is shown implanted in vessel 100. Endovascular bypass stent 104 comprises a main vessel stent graft 106 and a bypass stent graft 108. Main vessel stent graft 106 has an access port 110 located proximate clot 102. Bypass stent graft 108 has a proximate end 112 and a distal end 114. Proximate end 112 is connected to access port 110 in a sealing relationship, which will be explained further below with respect to FIG. 4. Distal end 114 resides within vessel 100 such that bypass catheter 108 bypasses clot 102 or other vascular pathology.

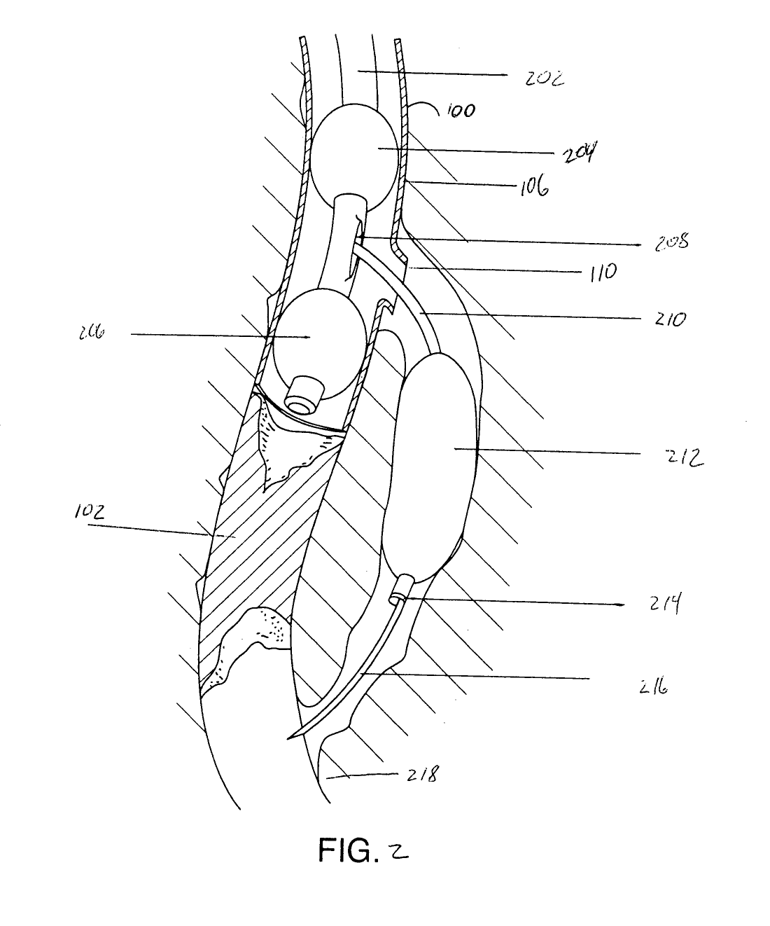

Implanting or deploying endovascular bypass stent 104 will be explained with reference to FIG. 2. First a main deployment catheter 202 and main vessel stent graft 106 are guided to clot 102 using standard endovascular surgical techniques. Main deployment catheter 202 comprises a proximate balloon 204, a distal balloon 206, and a working port 208. Inflating proximate balloon 204 and distal balloon 206 isolates working port 208 from blood flow.

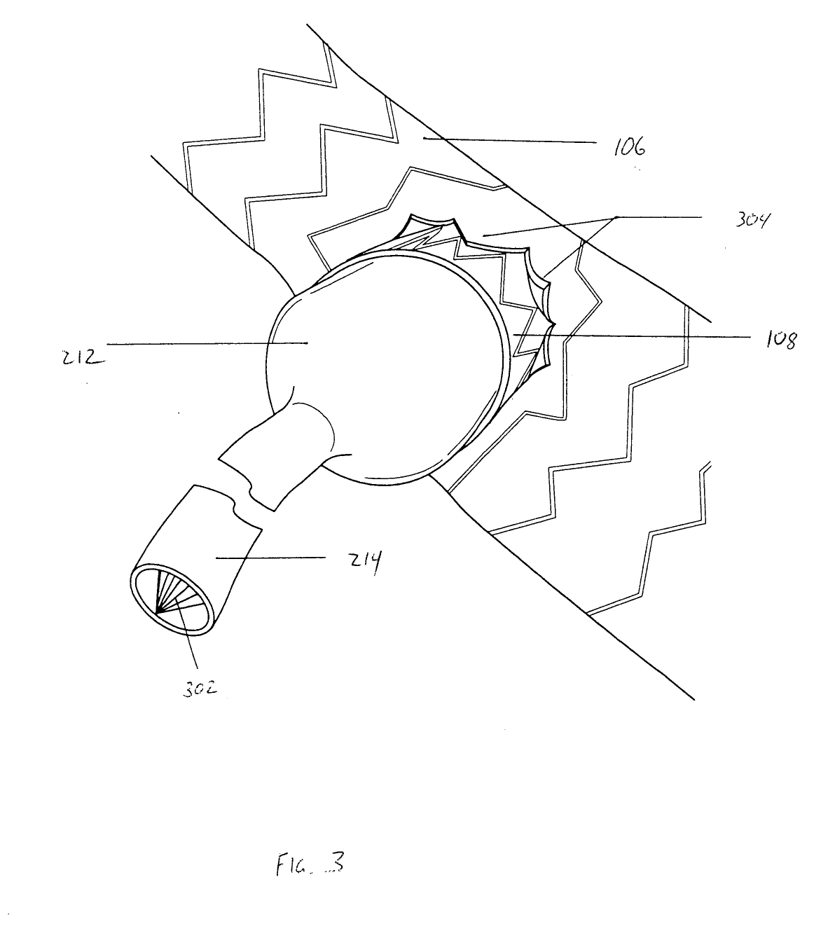

Referring now to FIG. 3, a trocar 302 is passed through the main deployment catheter 202 and out working port 208 once balloons 204 and 206 isolate blood flow. Using 3-D navigational technology (as is commonly available in the art), trocar 302 is aligned with working port 208 and used to puncture vessel 100 about working port 208. As shown in FIG. 3, main vessel stent graft 106 may be deployed without working port 208. In this case, trocar 302 first punctures main vessel stent graft 106 to make working port 208. When working port 208 is made by trocar 302, main vessel stent graft 106 is designed to form a controlled tear pattern, such as a controlled stellate pattern 304, as is commonly known in the art.

Once vessel 100 is punctured, a bypass catheter 210 is passed to the vascular pathology. Bypass catheter 210 comprises a dissecting balloon 212 and a tool port 214 at the distal end thereof. A wire needle 216 is passed out tool port 214. Using the bypass catheter 210, dissecting balloon 212 passes through the puncture and enters the perivascular space about vessel 100. The dissecting balloon dissects the perivascular space up to a vessel re-entry port 218. Vessel re-entry port 218 is shown as a part of blood vessel 100 such that clot 102 is removed from circulation, but vessel re-entry port 218 could reside in a separate blood vessel (not specifically shown) as required by the patient's anatomy and the particular pathology involved. Wire needle 216 punctures the vessel to establish re-entry port 218.

Once wire needle 216 establishes re-entry port 218, bypass catheter 210 is removed and bypass stent graft 108 is passed over wire needle 216. Distal end 114 is placed in the vessel at re-entry port 218 and expanded to fit snuggly with the vessel wall in a sealing relationship. Bypass stent graft could be expanded using a balloon or made out of an expanding material, such as, for example, shaped memory alloys. The proximate end 112 and access port 110 are joined in a sealing relationship, as explained below.

Once bypass stent graft 108 is placed, proximate balloon 204 is deflated and blood flow is verified. Finally, distal balloon 206 is deflated and the catheter is removed leaving endovascular bypass stent 104 in place.

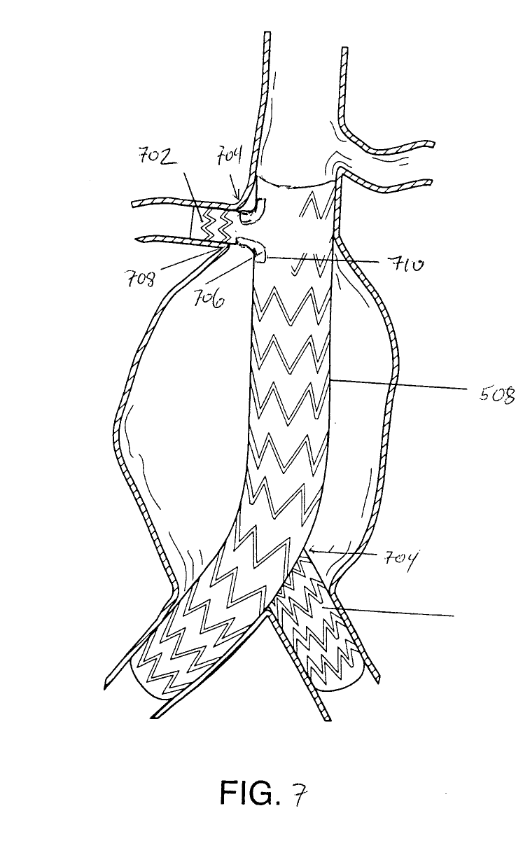

FIG. 4 shows the sealing relationship between access port 110 and proximate end 112 in more detail. In particular, a cross-sectional view of access port 110 and proximate end 112 is shown. Access port 110 has an edge 402 defining access port 110. About edge 402 is a seating surface 404. Proximate end 404 has a corresponding engaging surface 406. Engaging surface 406 mates with seating surface 404 to form a seal that inhibits blood leakage. Reference number 408 is a material that further inhibits bleeding. Reference number 408 could be a sealing ring, such as a GORTEX® washer, that could be deployed between seating surface 404 and engaging surface 406 to further inhibit blood flow. Alternatively, reference number 408 could be a form of epoxy, acrylic, silicone, tape, glue, or resin that seals seating surface 404 and engaging surface 406. Still further, bypass stent graft 108 and/or main vessel stent graft 102 could be constructed out of shaped memory alloys, such as, for example, Ag—Cd alloys, Cu—Al—Ni alloys, Cu—Sn alloys, Cu—Zn alloys, Cu—Zn—Si alloys, Cu—Zn—Sn alloys, Cu—Zn—Al alloys, In—Ti alloys, Ni—Al alloys, Ni—Ti alloys, Fe—Pt alloys, Mn—Cu alloys, Fe—Mn—Si alloys, and the like. These could be designed such that seating surface 404 and engaging surface 406 form an adequate seal and then deformed for deployment. After deployment, an activation signal could cause seating surface 404 and engaging surface 406 to join in a sealing relationship. The activation signal could be a thermal, electrical, magnetic, radiation signal or the like. Notice, the seal between access port 110 and bypass stent graft 108 could be accomplished using a connecting stent. Connecting stents are explained further below with reference to FIG. 7.

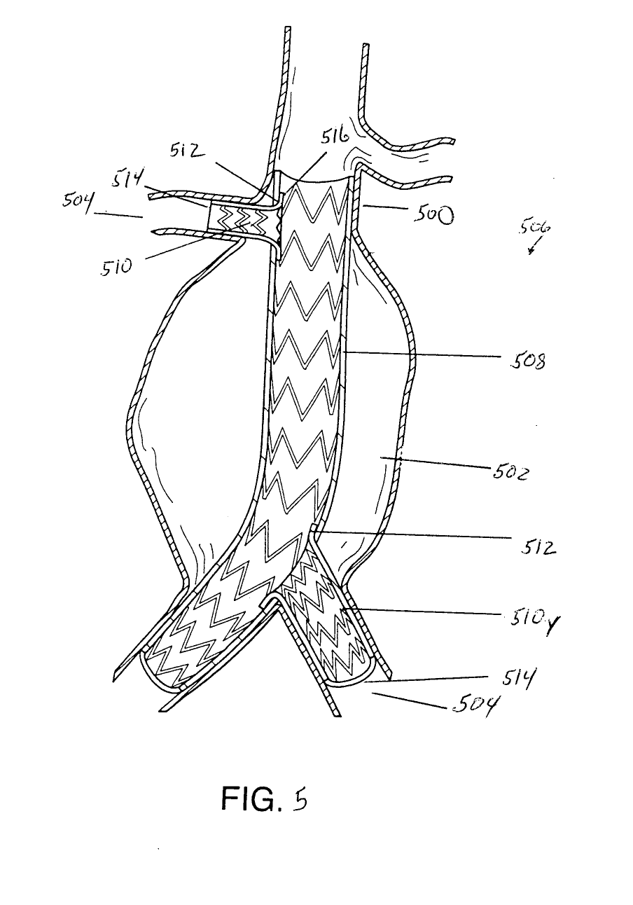

Referring now to FIG. 5, another embodiment of the present invention is shown. FIG. 5 shows a cut-away portion of a vessel 500. In this case, vessel 500 contains a type of aneurysm 502 or other vascular pathology that needs to be isolated from vessel 500. As shown, vessel 500 has branch vessels 504 that prevents the use of a conventional stent because a conventional stent would occlude blood flow to branch vessels 504 indefinitely. In this case, endovascular stent graft 506 includes a main vessel stent graft 508 and a number of branch connecting stent grafts 510. In this case, two branch connecting stent grafts 510 are shown, but more or less could be deployed as necessitated by patient anatomy. Branch connecting stent grafts 510 pass through access ports 512 in main vessel stent graft 508 such that a distal 514 of branch connecting stent graft 510 resides in branch vessels 504 and a proximate end 516 of branch connecting stent graft 510 is in a sealing relationship with access port 512, such sealing relationship is further explained above in connection with FIG. 4.

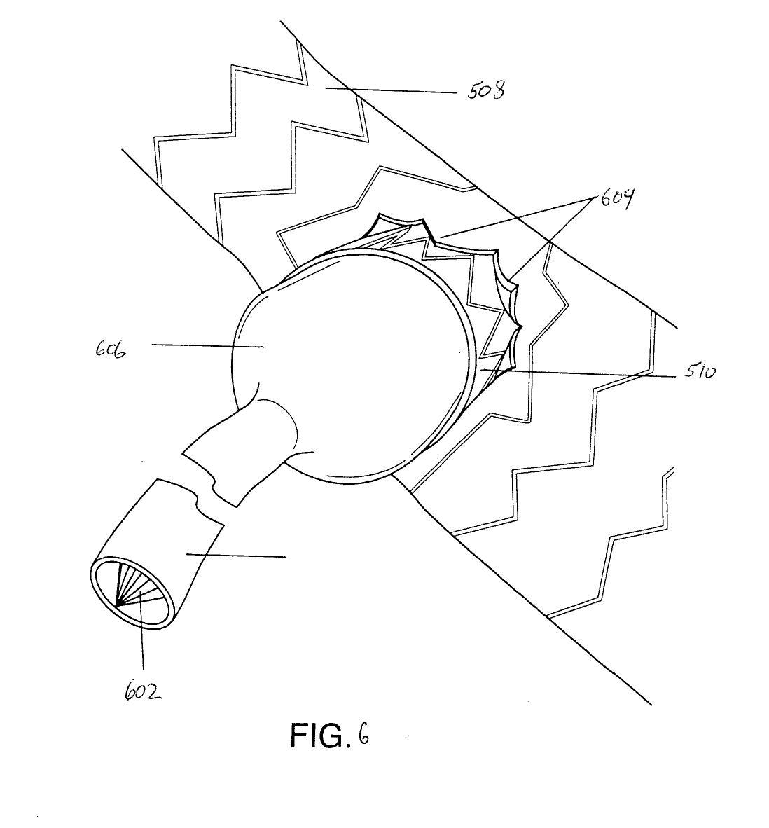

Endovascular stent graft 506 can be deployed in a number of different ways. For example, main vessel stent graft 508 can be placed using conventional endovascular techniques. Once placed, using 3-D surgical navigation techniques, commonly known in the art, a trocar 602 is used to puncture main vessel stent graft 508 at the junction with branch vessel 504 (See FIG. 6). Main vessel stent graft 508 is constructed such that trocar 602 would from a controlled tear 604, such as a controlled stellate pattern. A balloon 606 would be used to dilate tear 604 to a size capable of accepting branch connecting stent graft 510. Branch connecting stent graft 510 is the passed to the site such that distal end 514 resides in branch vessel 504 and proximate end 516 forms a sealing relationship with access port 512.

While main vessel stent graft 508 (and main vessel stent graft 106) is shown as a tubular member conforming to the shape of the vessel 500 (or 100), main vessel stent graft 508 could be other shapes, such as, for example, a y shaped main vessel stent graft 800. In this case, y branch 802 would replace branch connecting stent 510y (FIG. 5). Other shapes are possible.

Alternatively to using 3-D surgical navigation, FIG. 7 shows placing branch locating stent graft 702. Branch locating stent graft 702 would have a radiopaque edge 704 proximate vessel 500. Main vessel stent graft 508 would be passed to the vascular site occluding branch vessels 504. Trocar 604 would then be aligned with radiopaque edge 704 and main vessel stent graft 508 would be punctured to form access port 512. A connecting stent 706 would then be placed such that a distal end 708 of connecting stent 706 resided in and formed a sealing relationship with branch locating stent 702 and a proximate end 710 of connecting stent 706 resides in and forms a sealing relationship with access port 712.

While the invention has been particularly shown and described with reference to some embodiment thereof, it will be understood by those skilled in the art that various other changes in the form and details may be made without departing from the spirit and scope of the invention.

We claim:

1. A vascular stent graft for bypassing a portion of vascular anatomy, the vascular stent graft comprising:
a main vessel stent graft extending in the vessel until it is proximate the portion of the vascular anatomy to be bypassed;
a bypass stent graft comprising a proximate end and a distal end;
the proximate end of the bypass stent graft to be received in an access port on a wall of the main vessel stent graft in a sealing relationship; and
the distal end of the bypass stent graft to be positioned in a sealing relationship with the vessel such that the bypass stent graft bypasses the portion of vascular anatomy.

2. The vascular stent graft according to claim 1, wherein the access port on the wall of the main vessel stent graft is established by puncturing the wall of the main vessel stent graft and expanding the puncture in a controlled pattern.

3. The vascular stent graft according to claim 1, wherein
the access port is defined by an edge and comprises a seating surface about the edge, and
the proximate end of the bypass stent graft comprises an engaging surface, such that when the bypass stent graft is received in the access port, the seating surface and the engaging surface form a seal that inhibits blood flow.

4. The vascular stent graft according to claim 3, further comprising a material between seating surface and the engaging surface.

5. The vascular stent graft according to claim 4, wherein the material comprises at least one of a gasket, an epoxy, a resin, an acrylic, a silicone, a glue, and a tape.

6. The vascular stent graft according 3 wherein at least the proximate end of the bypass stent graft comprises a shaped memory alloy such that on activation, the engaging surface engages the seating surface in a sealing relationship.

7. The vascular stent graft according to claim 1, wherein the proximate end of the bypass stent graft resides in a first vessel and the distal end of the bypass stent graft resides in a second vessel.

8. The vascular stent graft according to claim 1, further comprising a branch connecting stent coupling the main vessel stent graft and the bypass stent graft, the branch connecting stent couples the main vessel stent graft and the bypass stent graft in the sealing relationship.

9. The vascular stent graft according to claim 3, wherein the proximate end of the bypass stent graft is flared.

10. A vascular stent graft for removing a portion of vascular anatomy from circulation but preserving circulation to branch vessels, the vascular stent graft comprising:
a main vessel stent graft; and
at least one branch connecting stent graft, wherein
each of the at least one branch connecting stent grafts to be received in an access port on a wall of the main vessel stent graft such that a distal end of the branch connecting stent graft will reside in a branch vessel and a proximate end of the branch connecting stent graft will form a sealing relationship with the access port.

11. The vascular stent graft according to claim 10, wherein
the access port on the wall of the main vessel stent graft is formed by puncturing the wall of the main vessel stent graft and expanding the tear in a controlled pattern.

12. The vascular stent graft according to claim 10, wherein
the access port is defined by an edge and a seating surface resides about the edge, and
the proximate end of the branch connecting stent graft comprising an engaging surface, such that when the branch connecting stent graft is received by the access port, the seating surface and engaging surface form the sealing relationship.

13. The vascular stent graft according to claim 10, wherein the at least one branch connecting stent graft comprises a plurality of branch connecting stent grafts.

14. The vascular stent graft according to claim 12, wherein at least the proximate end of the branch connecting stent graft comprises an expandable material such that the proximate end is expanded until the engaging surface and the seating surface form a sealing relationship.

15. The vascular stent graft according to claim 13, wherein the proximate end of the branch connecting stent graft is flared.

16. A vascular stent graft for removing a portion of vascular anatomy from circulation but preserving circulation to branch vessels, the vascular stent graft comprising:
a main vessel stent graft;
at least one branch stent graft; and
a corresponding number of connecting stents, wherein
the at least one branch stent graft comprises a distal end and a proximate end, the proximate end residing about a wall of the main vessel stent graft, the proximate end having at least one radiopaque marker;
the wall of the of the main vessel being designed to form an access port for each branch stent graft such that each of the access ports are aligned with proximate end, the alignment being identifiable by the at least one radiopaque marker;
each of the connecting stents having a main vessel seating surface and a branch vessel seating surface, such that the main vessel seating surface engages the wall of the main vessel stent graft in a sealing relationship and the branch vessel seating surface engages a wall of the branch stent graft in a sealing relationship.

17. The vascular graft stent according to claim 16 wherein the main vessel seating surface is flared.

18. The vascular graft stent according to claim 17 wherein the branch vessel seating surface is expandable such that the branch vessel seating surface is flush with the wall of the branch stent graft.

19. A vascular stent graft comprising:
a first stent graft;
the first stent graft comprises a port in a wall;
a second stent graft;
the second stent graft being received in the port in the wall; and
means for coupling the first stent graft and the second stent graft in a sealing relationship about the port.

20. The vascular stent graft according to claim 19, wherein the means for coupling includes at least one of a washer, an epoxy, a resin, a silicone, an acrylic, a glue, and a tape.
